(19) United States
(12) Patent Application Publication
(10) Pub. No.: US 2024/0303383 A1
(43) Pub. Date: Sep. 12, 2024

(54) ELECTRONIC DEVICE FOR OBTAINING COMMON SUPERCELL INFORMATION, AND ITS OPERATING METHOD

(71) Applicant: KOREA INSTITUTE OF SCIENCE AND TECHNOLOGY, Seoul (KR)

(72) Inventors: Jung-Hoon LEE, Seoul (KR); Weon Gyu LEE, Seoul (KR)

(21) Appl. No.: 18/492,365

(22) Filed: Oct. 23, 2023

(30) Foreign Application Priority Data
Mar. 9, 2023 (KR) 10-2023-0031029

(51) Int. Cl. G06F 30/10

(52) U.S. Cl. CPC G06F 30/10; G06F 2111/10

(57) ABSTRACT

An electronic device for obtaining common supercell information according to an embodiment of the inventive concept stores first and second lattice matrices, indicative of surface structures of two different materials in a real plane, and a conversion table. The processor retrieves these matrices, obtains complex lattice matrices for both surfaces in a complex plane, and determines a lattice constant ratio from them. Using this ratio, the processor refers to the conversion table to obtain phase information and a conversion matrix. Finally, it calculates common supercell information for both materials based on the lattice constant ratio, phase information, and conversion matrix, and outputs it via the user interface device.

### CROSS-REFERENCE TO RELATED APPLICATION

This U.S. non-provisional patent application claims priority under 35 U.S.C. § 119 of Korean Patent Application No. 10-2023-0031029, filed on Mar. 9, 2023, the entire contents of which are hereby incorporated by reference.

### BACKGROUND

The present disclosure herein relates to an electronic device for obtaining common supercell information, and its operating method.

A crystalline solid material has repeating unit cells. When attempting to match two different crystalline solid materials, it is not straightforward due to the different repetition periods of the two crystalline materials. In this case, matching the two crystal structures can be facilitated by finding a common supercell having the same periodicity between two crystal structures.

Meanwhile, it is rare for the periodicity of two different crystal structures to perfectly coincide. Therefore, it is necessary to find common supercells within a certain tolerance range for lattice strain. Since it takes a lot of time to determine a common supercell by using conventional (manual) methods, there is a demand for electronic devices and analysis methods that are capable of assisting with or automatically determining the common supercell.

### SUMMARY

The present disclosure provides a method for effectively determining a common supercell by using an electronic device.

The present disclosure is not limited to the above, and others not mentioned will be clearly understood by those skilled in the art from the following description.

In an embodiment of the inventive concept, a method for operating an electronic device for obtaining common supercell information includes: receiving a first lattice matrix indicative of a first surface structure of a first material in a real plane; obtaining a first complex lattice matrix corresponding to the first surface structure in a complex plane on the basis of the first lattice matrix; receiving a second lattice matrix indicative of a second surface structure of a second material different from the first material in the real plane; obtaining a second complex lattice matrix corresponding to the second surface structure in the complex plane on the basis of the second lattice matrix; obtaining a lattice constant ratio on the basis of the first complex lattice matrix and the second complex lattice matrix; obtaining phase information corresponding to the lattice constant ratio and a conversion matrix corresponding to the lattice constant ratio by referring to a conversion table in the electronic device; and obtaining common supercell information of the first material and the second material on the basis of the lattice constant ratio, the phase information, and the conversion matrix.

In an embodiment, a relationship between the first complex lattice matrix and the second complex lattice matrix may be defined by an eigenvalue, and the eigenvalue may include the lattice constant ratio and the phase information.

In an embodiment, the lattice constant ratio may be a ratio of the second complex lattice matrix to the first complex lattice matrix, and the phase information may include a value obtained by subtracting a first phase value of the first complex lattice matrix from a second phase value of the second complex lattice matrix.

In an embodiment, each of the first material and the second material may have a cubic structure, the lattice constant ratio may be √(m²+n²), the phase information may be arctan(n/m), and the conversion matrix may include a first conversion element value (e₁₁), a second conversion element value (e₁₂), a third conversion element value (e₂₁), and a fourth conversion element value (e₂₂), the first to fourth conversion element values (e₁₁, e₁₂, e₂₁, e₂₂) being respectively m, n, −n, and m, where n and m are arbitrary integers.

In an embodiment, each of the first material and the second material may have a hexagonal structure, the lattice constant ratio may be √(m²−mn+n²), the phase information may be arctan((√(3)*n)/(2m−n)), and the conversion matrix may include a first conversion element value (e₁₁), a second conversion element value (e₁₂), a third conversion element value (e₂₁), and a fourth conversion element value (e₂₂), the first to fourth conversion element values (e₁₁, e₁₂, e₂₁, e₂₂) being respectively m, n, −n, and m−n, where n and m are arbitrary integers.

In an embodiment, the obtaining of the phase information corresponding to the lattice constant ratio and the conversion matrix corresponding to the lattice constant ratio by referring to the conversion table may include: searching for a target lattice constant ratio closest to the lattice constant ratio among a plurality of lattice constant ratios stored in the conversion table; obtaining target phase information corresponding to the target lattice constant ratio as the phase information by referring to the conversion table; and obtaining a target conversion matrix corresponding to the target lattice constant ratio as the conversion matrix by referring to the conversion table.

In an embodiment, the conversion table may be implemented as a table in the form of a Python list.

In an embodiment, the obtaining of the common supercell information of the first material and the second material on the basis of the lattice constant ratio, the phase information, and the conversion matrix may include obtaining the common supercell information of the first material and the second material on the basis of the lattice constant ratio, the phase information, and the conversion matrix by using a numpy library and a pymatgen library of Python.

In an embodiment of the inventive concept, an electronic device for obtaining common supercell information includes: a memory device configured to store a first lattice matrix indicative of a first surface structure of a first material in a real plane, a second lattice matrix indicative of a second surface structure of a second material different from the first material in the real plane, and a conversion table; a processor; and a user interface device, wherein the processor is configured to: load the first lattice matrix and the second lattice matrix from the memory device; obtain a first complex lattice matrix corresponding to the first surface structure in a complex plane on the basis of the first lattice matrix; obtain a second complex lattice matrix corresponding to the second surface structure in the complex plane on the basis of the second lattice matrix; obtain a lattice constant ratio on the basis of the first complex lattice matrix and the second complex lattice matrix; obtain phase information corresponding to the lattice constant ratio and a conversion matrix corresponding to the lattice constant ratio by referring to the conversion table; obtain common supercell information of the first material and the second material on the basis of the lattice constant ratio, the phase information, and the conversion matrix; and output the common supercell information through the user interface device.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept.

In the drawings:

FIG. 1 is a block diagram of an electronic device according to an embodiment of the inventive concept;

FIG. 2 is a flowchart illustrating a method for operating an electronic device according to an embodiment;

FIG. 3 shows a first complex lattice matrix and a second complex lattice matrix disposed on a complex plane;

FIG. 4 is a flowchart illustrating a method for operating an electronic device according to an embodiment;

FIG. 5 is a diagram illustrating an exemplary conversion table;

FIG. 6 is a flowchart illustrating a method for operating an electronic device according to an embodiment; and

FIG. 7 is a graph illustrating a relationship between search ranges and time costs in an embodiment and a comparative example.

### DETAILED DESCRIPTION

Embodiments of the inventive concept will be described with reference to the accompanying drawings to fully understand the configuration and effects of the inventive concept. The inventive concept, however, is not limited to the embodiments disclosed below, and may be implemented in various forms and various changes may be applied. Rather, these embodiments are provided so that this disclosure will be complete, and will fully convey the scope of the inventive concept to those skilled in the art. In the accompanying drawings, for convenience of explanation, the size of components is shown larger than the actual size, and the ratio of each component may be exaggerated or reduced.

A common supercell refers to a cell determined by using multiples of vectors constituting a first unit cell of a first material and multiples of vectors constituting a second unit cell of a second material. In this specification, common supercell information refers to vectors constituting a common supercell.

Vectors indicating the common supercell may not completely match multiples of vectors constituting the first unit cell and multiples of vectors constituting the second unit cell of the second material. Vectors constituting the common supercell may be similar to multiples of vectors constituting the first unit cell and multiples of vectors constituting the second unit cell of the second material.

An embodiment of the inventive concept proposes a method for effectively determining a common supercell of two materials by using an electronic device.

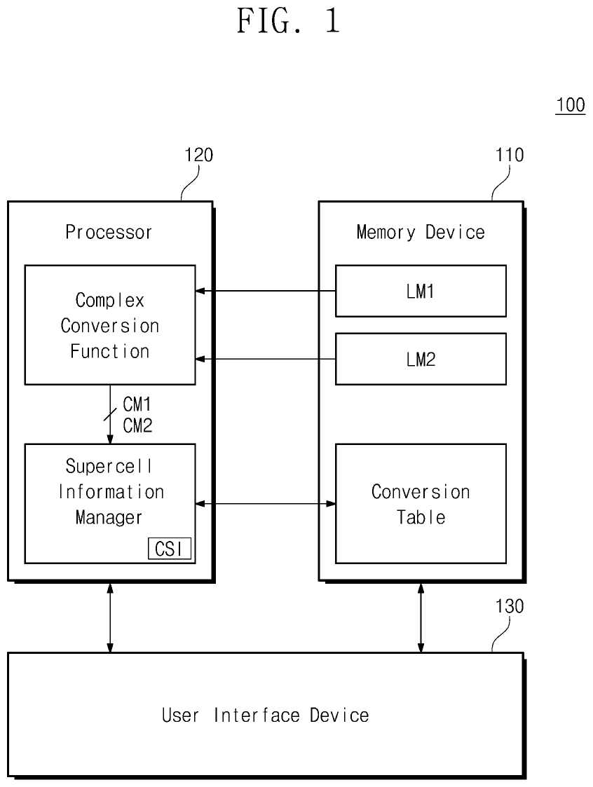

FIG. 1 is a block diagram of an electronic device according to an embodiment of the inventive concept.

Referring to FIG. 1, an electronic device 100 may include a memory device 110, a processor 120, and a user interface device 130. The electronic device 100 may be a device that calculates common supercell information (CSI) of different materials. For example, the electronic device 100 may be implemented as a PC, laptop, tablet PC, smart phone, server device, or the like.

The memory device 110 may store programs for processing and control of the processor 120 and may store data input to or output from the electronic device 100. The memory device 110 may include a temporary memory (e.g., random access memory (RAM), buffer, etc.) or a non-temporary memory (e.g., storage such as a magnetic disk). The memory device 110 may include at least one type of storage medium among a flash memory-type, hard disk-type, multimedia card micro-type, or card-type memory (e.g., SD or XD memory, etc.), random access memory (RAM), static random access memory (SRAM), read-only memory (ROM), electrically erasable programmable read-only memory (EEPROM), programmable read-only memory (PROM), magnetic memory, magnetic disc, or optical disc.

The memory device 110 may store a first lattice matrix LM1, a second lattice matrix LM2, and a conversion table. The first lattice matrix LM1 may indicate a first surface structure of a first material in a real plane. The first lattice matrix LM1 may include vectors constituting a first unit cell of the first material. The second lattice matrix LM2 may indicate a second surface structure of a second material different from the first material in the real plane. The second lattice matrix LM2 may include vectors constituting a second unit cell of the second material. The first surface structure of the first material and the second surface structure of the second material may have similar lattices.

For example, the first surface structure of the first material may have a cubic lattice, and the second surface structure of the second material may also have a cubic lattice. As another example, the first surface structure of the first material may have a hexagonal lattice, and the second surface structure of the second material may also have a hexagonal lattice.

The memory device 110 may provide information on the first lattice matrix LM1, the second lattice matrix LM2, or the conversion table to the processor 120 under the control of the processor 120. Otherwise, the memory device 110 may transmit and receive information of a first lattice matrix LM1, a second lattice matrix LM2, or a conversion table from the outside or from the processor 120 through the user interface device 130.

According to some embodiments, the first lattice matrix LM1 may be obtained from a CIF file of the first material. The second lattice matrix LM2 may be obtained from a CIF file of the second material.

The processor 120 may control overall operations of the electronic device 100. For example, the processor 120 may store data in the memory device 110, read data stored in the memory device 110, or process a request from the outside (e.g., the electronic device 100) through the user interface device 130.

The processor 120 may include a complex conversion function and a supercell information manager. The complex conversion function and the supercell information manager may be implemented in hardware, software, or a combination of hardware and software.

The complex conversion function may receive the first lattice matrix LM1 and the second lattice matrix LM2 from the memory device 110. The complex conversion function may generate a first complex lattice matrix CM1 that corresponds to the first lattice matrix LM1 and includes vectors on a complex plane on the basis of the first lattice matrix LM1 on a real plane. Similarly, the complex conversion function may generate a second complex lattice matrix CM2 that corresponds to the second lattice matrix LM2 and includes vectors on a complex plane on the basis of the second lattice matrix LM2 on a real plane. The complex conversion function may provide information on the first complex lattice matrix CM1 and the second complex lattice matrix CM2 to the supercell information manager.

The supercell information manager may generate a lattice constant ratio on the basis of the first complex lattice matrix CM1 and the second complex lattice matrix CM2. The lattice constant ratio may indicate a ratio between the size value of a vector of the first complex lattice matrix CM1 and the size value of a vector of the second complex lattice matrix CM2.

The supercell information manager may refer to a conversion table in the memory device 110 on the basis of the lattice constant ratio and obtain phase information and a conversion matrix corresponding to the lattice constant ratio. The phase information may indicate a difference between a phase change value when converting the first complex lattice matrix CM1 to a common supercell and a phase change value when converting the second complex lattice matrix CM2 to a common supercell.

The conversion matrix may be used to obtain common supercell information CSI from the first complex lattice matrix CM1 and the second complex lattice matrix CM2. Otherwise, the conversion matrix may be used to obtain the second complex lattice matrix CM2 from the first complex lattice matrix CM1, or may be used to obtain the first complex lattice matrix CM1 from the second complex lattice matrix CM2.

The supercell information manager may generate common supercell information (CSI) on the basis of the lattice constant ratio, the phase information, and the conversion matrix. The common supercell information (CSI) may indicate information on a common supercell of the first material and the second material. The supercell information manager may provide the common supercell information (CSI) to the user interface device 130.

According to some embodiments, the supercell information manager may generate a CIF file of a common supercell by using the common supercell information (CSI).

The user interface device 130 may provide an interface between the electronic device 100 and a user. The user interface device 130 may communicate with the memory device 110 and the processor 120. The user interface device 130 may provide, to the memory device 110, data (e.g., information of the first lattice matrix LM1, the second lattice matrix LM2, and the conversion table, etc.) received from a user or a separate external electronic device. The user interface device 130 may provide the common supercell information (CSI) received from the processor 120 in a visual form to a user (e.g., may display the common supercell information (CSI) on a display device).

For example, the user interface device 130 may be implemented as a display device, a touch screen, a mouse, a keyboard, or the like.

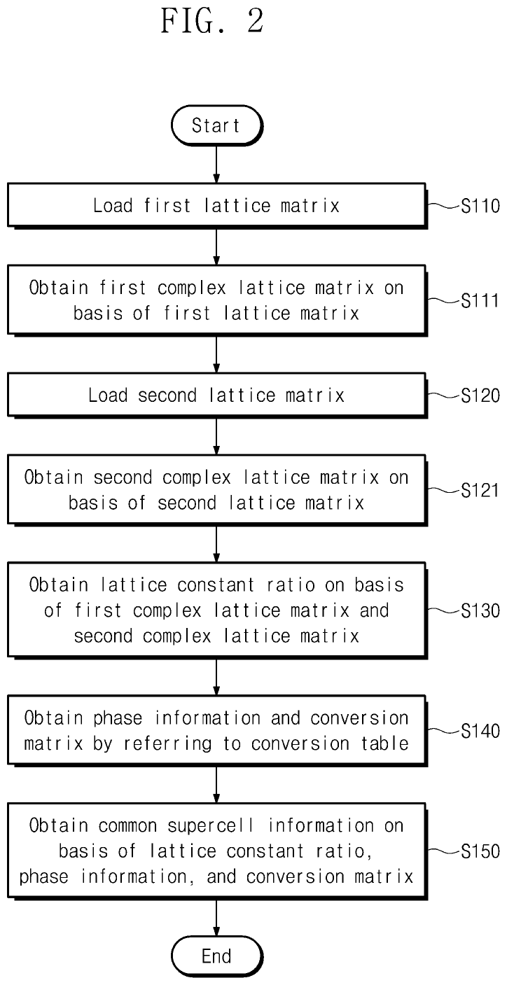

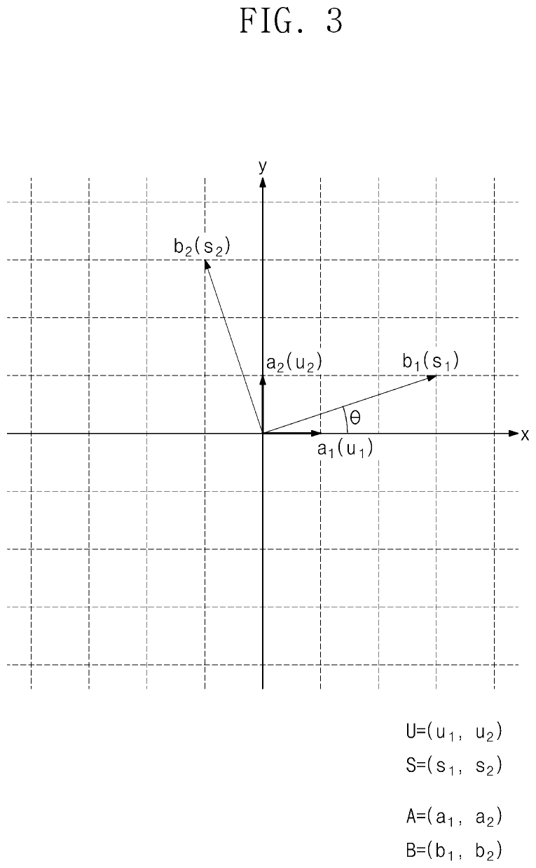

FIG. 2 is a flowchart illustrating a method for operating an electronic device according to an embodiment. FIG. 3 shows a first complex lattice matrix and a second complex lattice matrix disposed on a complex plane. Referring to FIGS. 1 and 2, a method for operating an electronic device will be described.

In S110, the electronic device 100 may load a first lattice matrix. The first lattice matrix may indicate a first surface structure of a first material. In FIG. 3, when an x-y plane is a real plane indicating real number-real number, a first lattice matrix U on a real plane may be represented by vectors u₁, u₂.

In S111, the electronic device 100 may obtain a first complex lattice matrix corresponding to the first surface structure in a complex plane on the basis of the first lattice matrix. The first complex lattice matrix may be a value obtained by making the first lattice matrix correspond to a complex plane.

In FIG. 3, when an x-y plane is a complex plane, a first complex lattice matrix A may be represented as complex number vectors a₁, a₂. In the case of a complex plane, in FIG. 3, a component corresponding to an x-axis indicates a real part, and a component corresponding to a y-axis indicates an imaginary part.

For example, as shown in FIG. 3, when the vectors of the first lattice matrix U are u₁=(1, 0) and u₂=(0, 1), the vectors of the first complex lattice matrix in a complex plane may be a₁=1 and a₂=i.

In S120, the electronic device 100 may load a second lattice matrix. The second lattice matrix may indicate a second surface structure of a second material in a real plane. In FIG. 3, when an x-y plane is a real plane indicating real number-real number, a second lattice matrix S in a real plane may be expressed as vectors s₁, s₂.

In S121, the electronic device 100 may obtain a second complex lattice matrix corresponding to the second surface structure in a complex plane on the basis of the second lattice matrix. The second complex lattice matrix may be a value obtained by making the second lattice matrix correspond to a complex plane. In FIG. 3, when an x-y plane is a complex plane indicating real number-imaginary number, a second complex lattice matrix B may be expressed as vectors b₁, b₂.

For example, as shown in FIG. 3, when the vectors of the second lattice matrix S are s₁=(3, 1), s₂=(−1, 3), the vectors of the second complex lattice matrix B in a complex plane may be b₁=3+i, b₂=−1+3i.

In S130, a lattice constant ratio may be obtained on the basis of the first complex lattice matrix and the second complex lattice matrix. The lattice constant ratio r may be a ratio between the size of vectors constituting the first complex lattice matrix and the size of vectors constituting the second complex lattice matrix. The lattice constant ratio r may be expressed as Equation 1 below.

$$r = \vert {b}_{1} \vert / \vert {a}_{1} \vert \qquad [ Equation 1 ]$$

In an embodiment of the inventive concept, when the first material has a cubic lattice or a hexagonal lattice, the vector a₁ and the vector a₂ have the same size. When the second material has a cubic or hexagonal lattice, the vector b₁ and the vector b₂ have the same size.

In S140, the electronic device 100 may obtain phase information and a conversion matrix by referring to a conversion table.

For example, as shown in FIG. 3, when the vectors of the first complex lattice matrix A are a₁=1, a₂=i, the common supercell information of the first and second materials that are similar may correspond to the second complex lattice matrix. For example, as shown in Equation 2 below, the first complex lattice matrix A may be calculated as the second complex lattice matrix B through a conversion matrix T.

$$TA = B \qquad [ Equation 2 ]$$

Here, the first complex lattice matrix may indicate an eigen vector. Rewriting Equation 2, a value obtained by calculating the conversion matrix T on the first complex lattice matrix A is equivalent to a value obtained by multiplying the first complex lattice matrix A by a constant c as shown in Equation 3 below, or a value obtained by multiplying the first complex lattice A by re^{iθ}.

$$TA = c A = r {e}^{i θ} A \qquad [ Equation 3 ]$$

Here, re^{iθ} corresponds to an eigen value.

θ of Equation 3 corresponds to phase information, and the phase information θ corresponds to a value obtained by subtracting a first phase change value when the first complex lattice matrix A is converted from a phase change value when the second complex lattice matrix B is converted. Elements of the conversion matrix T and the phase information θ corresponding to the lattice constant ratio r are deterministically stored in the conversion table. Elements of the conversion matrix T may include (n, m).

For example, when both the first surface structure of the first material and the second surface structure of the second material have a cubic lattice, the conversion matrix T may include a first conversion element value (e₁₁), a second conversion element value (e₁₂), a third conversion element value (e₂₁), and a fourth conversion element value (e₂₂), wherein the first to fourth conversion element values (e₁₁, e₁₂, e₂₁, e₂₂) are respectively m, n, −n, m. The first to fourth conversion element values (e₁₁, e₁₂, e₂₁, e₂₂) may be expressed as a first element n and/or a second element m. The first element n and the second element m may be arbitrary integers.

$$T = [ \begin{matrix}m & n \\ - u & m\end{matrix} ] \qquad [ Equation 4 ]$$

The lattice constant ratio r and the first and second elements n, m of the conversion matrix T may have a relationship as shown in Equation 5 below.

$$r = √ ( {m}^{2} + {n}^{2} ) \qquad [ Equation 5 ]$$

Elements of the conversion matrix T and the phase information θ may have a relationship as shown in Equation 6 below.

$$θ = arctan ( n / m ) \qquad [ Equation 6 ]$$

Compared to when the first surface structure of the first material and the second surface structure of the second material both have a cubic lattice, both the first surface structure of the first material and the second surface structure of the second material may have a hexagonal lattice.

In this case, the conversion matrix T may include the first conversion element value (e₁₁), the second conversion element value (e₁₂), the third conversion element value (e₂₁), and the fourth conversion element value (e₂₂) as shown in Equation 7 below, wherein the first to fourth conversion element values (e₁₁, e₁₂, e₂₁, e₂₂) are respectively m, n, −n, and m−n. The first to fourth conversion element values (e₁₁, e₁₂, e₂₁, e₂₂) may be expressed as a first element n and/or a second element m. The first element n and the second element m may be arbitrary integers.

$$T = [ \begin{matrix}m & n \\ - u & m - n\end{matrix} ] \qquad [ Equation 7 ]$$

Elements of the conversion matrix T and the lattice constant ratio r may have a relationship as shown in Equation 8 below.

$$√ ( {m}^{2} - mn + {n}^{2} ) \qquad [ Equation 8 ]$$

Elements of the conversion matrix T and the phase information θ may have a relationship as shown in Equation 9 below.

$$arctan ( ( √ ( 3 ) * n ) / ( 2 m - n ) ) \qquad [ Equation 9 ]$$

According to an embodiment of the inventive concept, when the surface structure of the first material and the surface structure of the second material resemble each other, the first element n and the second element m of the conversion matrix may be obtained through the conversion table if the lattice constant ratio r is known. When the first element n and the second element m are known, the phase information θ and the conversion matrix T may be found immediately. As a result, by using Equation 3, common supercell information may be deterministically found by calculating the constant c or the conversion matrix T in the first complex lattice matrix A.

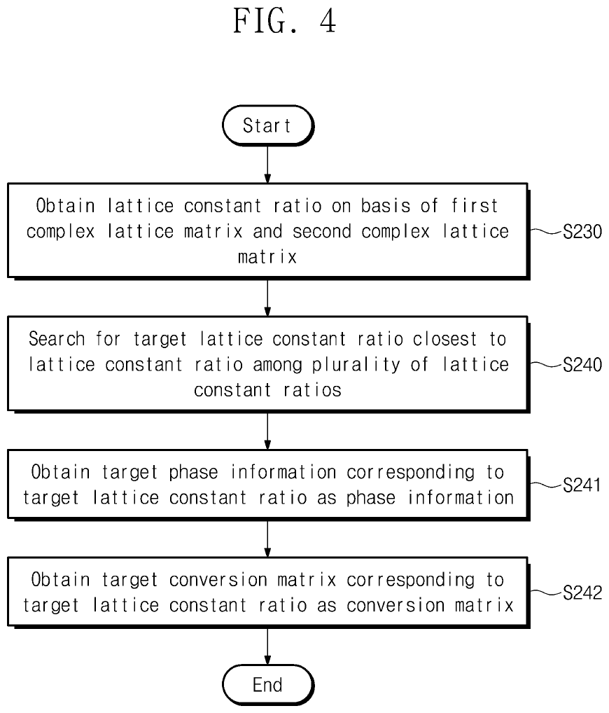

FIG. 4 is a flowchart illustrating a method for operating an electronic device according to an embodiment. Referring to FIG. 4, a method for operating an electronic device will be described. The electronic device may correspond to the electronic device of FIG. 1. The method of FIG. 4 may correspond to S130 and S140 of FIG. 3. The electronic device 100 may obtain a first complex lattice matrix on the basis of a first lattice matrix, and obtain a second complex lattice matrix on the basis of a second lattice matrix. The electronic device may include a processor and a conversion table.

In S230, the electronic device may obtain a lattice constant ratio on the basis of the first complex lattice matrix and the second complex lattice matrix.

In S240, the electronic device may search for a target lattice constant ratio closest to the lattice constant ratio among a plurality of lattice constant ratios. More specifically, the processor may refer to a conversion table. The conversion table may store a plurality of lattice constant ratios. The processor may determine, as a target lattice constant ratio, the target lattice constant ratio closest to the lattice constant ratio of S230 among a plurality of lattice constant ratios in the conversion table. The processor may load (e.g., buffer in a cache memory of the processor) the target lattice constant ratio.

In S241, the electronic device may obtain, as phase information, target phase information corresponding to the target lattice constant ratio.

In S242, the electronic device may obtain, as a conversion matrix, a target conversion matrix corresponding to the target lattice constant ratio.

More specifically, the conversion table may store a plurality of sets composed of a lattice constant ratio r, phase information, and a conversion matrix. The processor may determine, as a target lattice constant ratio, the target lattice constant ratio closest to the lattice constant ratio among a plurality of lattice constant ratios in the conversion table, and load the target phase information and target conversion matrix corresponding to the target lattice constant ratio in the conversion table. The processor may determine the target phase information as phase information corresponding to the lattice constant ratio of S230. The processor may obtain the target conversion matrix as a conversion matrix corresponding to the lattice constant ratio of S230.

As described above, the electronic device may search for a close value among a plurality of sets of the lattice constant ratio, phase information, and conversion matrix previously learned in the conversion table.

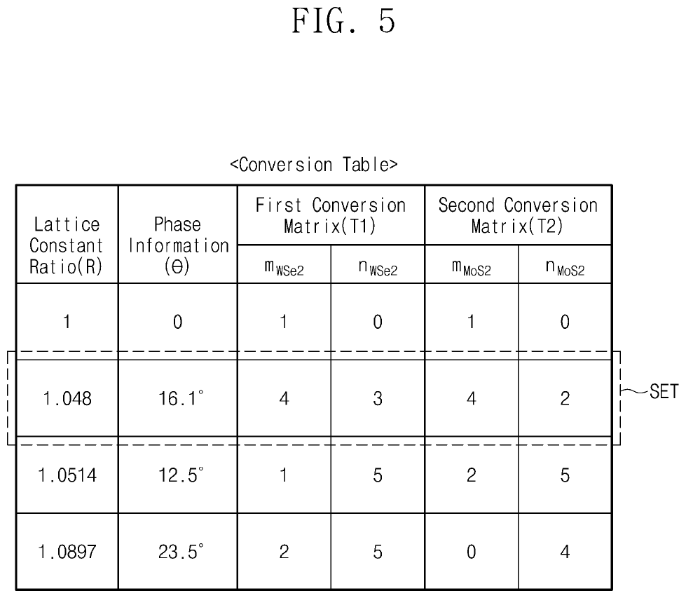

FIG. 5 is a diagram illustrating an exemplary conversion table.

Specifically, FIG. 5 is a diagram showing a conversion table used in a process of finding a common supercell of tungsten diselenide (WSe₂) and molybdenum disulfide (MoS₂). The conversion table may include a plurality of sets of lattice constant ratio r, phase information θ, a first conversion matrix T1, and a second conversion matrix T2.

For example, a first material may be tungsten diselenide (WSe₂), and a second material may be molybdenum disulfide (MoS₂). Each of crystal structures of tungsten diselenide (WSe₂) and molybdenum disulfide (MoS₂) may be a hexagonal lattice. The lattice constant of tungsten diselenide (WSe₂) may be 3.282 Å, and the lattice constant of molybdenum disulfide (MoS₂) may be 1.038 Å. A lattice constant ratio r of tungsten diselenide (WSe₂) and molybdenum disulfide (MoS₂) may be about 1.038. (r=3.282 Å/3.138 Å=1.038)

A target lattice constant ratio closest to 1.038 may be searched among a plurality of lattice constant ratios r in the conversion table. Target phase information corresponding to the target lattice constant ratio may be obtained as phase information θ. A target conversion matrix corresponding to the target lattice constant ratio may be obtained as a conversion matrix.

Here, when the complex lattice matrix of tungsten diselenide (WSe₂) becomes an eigen vector, a first element (n_{WSe2}) and a second element (m_{WSe2}) of the first conversion matrix T1 corresponding thereto may be found. In the case of substituting the value of the first element (n_{WSe2}) and the value of the second element (m_{WSe2}) into Equation 7 and calculating the complex lattice matrix of tungsten diselenide (WSe₂) with the conversion matrix, common supercell information of tungsten diselenide (WSe₂) and molybdenum disulfide (MoS₂) may be obtained.

In addition, when the complex lattice matrix of molybdenum disulfide (MoS₂) becomes an eigen vector, a first element (n_{MoS2}) and a second element (m_{MoS2}) of the second conversion matrix T2 corresponding thereto may be found. In the case of substituting the value of the first element (n_{MoS2}) and the value of the second element (m_{Mos2}) into Equation 7 and calculating the complex lattice matrix of molybdenum disulfide (MoS₂) with the conversion matrix, common supercell information of tungsten diselenide (WSe₂) and molybdenum disulfide (MoS₂) may be obtained.

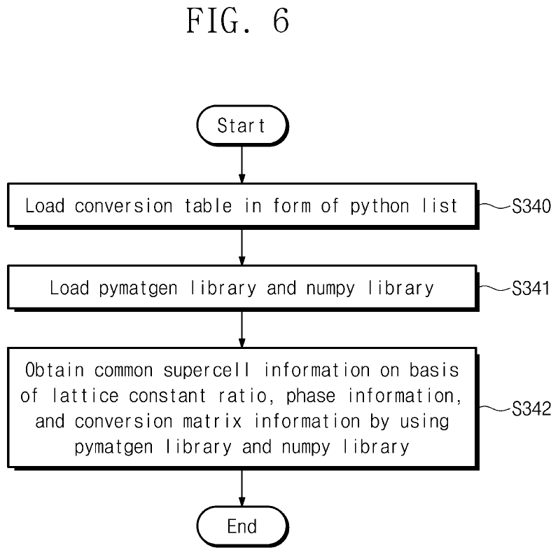

FIG. 6 is a flowchart illustrating a method for operating an electronic device according to an embodiment. Referring to FIG. 6, a method for operating an electronic device will be described. The electronic device may correspond to the electronic device of FIG. 1. The method of FIG. 6 may correspond to S140 and S150 of FIG. 3.

In S340, the electronic device 100 may load a conversion table in the form of a Python list.

In S341, the electronic device 100 may load a Pymatgen library and a numpy library.

In S342, the electronic device 100 may obtain common supercell information on the basis of a lattice constant ratio, phase information, and conversion matrix information by using the Pymatgen library and the numpy library.

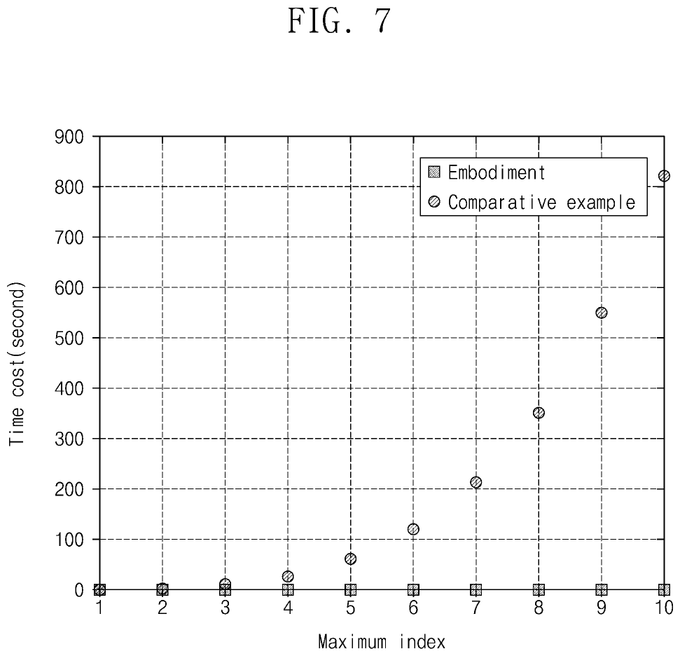

FIG. 7 is a graph illustrating a relationship between search ranges and time costs in an embodiment and a comparative example. Common supercell information of a first material and a second material having resembling lattice structures may be found in both a comparative example and an embodiment.

Unlike an embodiment of the inventive concept, in a method for operating an electronic device in a comparative example, all possible common supercell information is found on the basis of a first lattice matrix indicating a first surface structure of a first material in a real plane and a second lattice matrix indicating a second surface structure of a second material in a real plane.

Referring to FIG. 7, since all possible two-dimensional common supercells are searched for, all four conversion elements of a conversion matrix for conversion to common supercells must be searched. In this case, in the case of finding a common supercell with a very large size, the time cost increases polynomially.

Unlike a comparative example, in an embodiment of the inventive concept, a first lattice matrix is converted to a first complex lattice matrix, and a second lattice matrix is converted to a second complex lattice matrix, and thus the number of elements may be reduced compared to the first lattice matrix and the second lattice matrix. In addition, by obtaining phase information and a conversion matrix by referring to a lattice constant ratio and a conversion table in an electronic device, the time required to acquire common supercell information of a first material and a second material may be greatly reduced compared to a comparative example.

By using an electronic device according to an embodiment of the inventive concept, a common supercell between a first crystal structure of a first material and a second crystal structure resembling a first crystal structure of a second material different from the first material may be easily determined.

Although the embodiments of the inventive concept have been described with reference to the accompanying drawings, the inventive concept may be implemented in other specific forms without changing the technical spirit or essential features. Therefore, it should be understood that the embodiments described above are illustrative in all respects and not limitative.

What is claimed is:

1. A method for operating an electronic device, the method comprising:
receiving a first lattice matrix corresponding to a first surface structure of a first material in a real plane;
obtaining a first complex lattice matrix corresponding to the first surface structure in a complex plane for the given first lattice matrix;
receiving a second lattice matrix corresponding to the second surface structure of a second material different from the first material in the real plane;
obtaining a second complex lattice matrix corresponding to the second surface structure in the complex plane for the given second lattice matrix;
obtaining a lattice constant ratio for the given first and second complex lattices;
obtaining phase information and a conversion matrix for given a lattice constant ratio by referring to a conversion table in the electronic device; and
obtaining common supercell information between the first and the second materials for the given the lattice constant ratio, the phase information, and the conversion matrix.

2. The method of claim 1, wherein the first surface structure of the first material and the second surface structure of the second material have a resemblance relationship.

3. The method of claim 2, wherein a relationship between the first and second complex lattice matrices is defined by an eigenvalue, and the eigenvalue includes the lattice constant ratio and the phase information.

4. The method of claim 1, wherein the lattice constant ratio is a ratio of the second complex lattice matrix to the first complex lattice matrix, and the phase information includes a value obtained by subtracting a first phase value of the first complex lattice matrix from a second phase value of the second complex lattice matrix.

5. The method of claim 1, wherein the first and the second materials have cubic structures, the lattice constant ratio is √(m²+n²), the phase information is arctan(n/m), and the conversion matrix includes a first conversion element value (e₁₁), a second conversion element value (e₁₂), a third conversion element value (e₂₁), and a fourth conversion element value (e₂₂), the first to fourth conversion element values (e₁₁, e₁₂, e₂₁, e₂₂) correspond to m, n, −n, and m, respectively, where n and m are arbitrary integers.

6. The method of claim 1, wherein the first and the second materials have a hexagonal structure, the lattice constant ratio is √(m²−mn+n²), the phase information is arctan((√(3)*n)/(2m−n)), and the conversion matrix includes a first conversion element value (e₁₁), a second conversion element value (e₁₂), a third conversion element value (e₂₁), and a fourth conversion element value (e₂₂), the first to fourth conversion element values (e₁₁, e₁₂, e₂₁, e₂₂) correspond to m, n, −n, and m−n, respectively, where n and m are arbitrary integers.

7. The method of claim 1, wherein the obtaining of the phase information corresponding to the lattice constant ratio and of the conversion matrix corresponding to the lattice constant ratio from the conversion table includes:
searching for a target lattice constant ratio closest to the lattice constant ratio among lattice constant ratios stored in the conversion table;
obtaining target phase information corresponding to the target lattice constant ratio as the phase information from the conversion table; and
obtaining a target conversion matrix corresponding to the target lattice constant ratio as the conversion matrix from the conversion table.

8. The method of claim 1, wherein the conversion table is implemented as a table with the form of a Python list.

9. The method of claim 1, wherein the obtaining of the common supercell information between the first and second materials for the given lattice constant ratio, the phase information, and the conversion matrix includes obtaining the common supercell information of the first material and the second material for the given lattice constant ratio, the phase information, and the conversion matrix by using a numpy library and a pymatgen library of Python.

10. An electronic device comprising:
a memory device configured to store a first lattice matrix corresponding to a first surface structure of a first material in a real plane, a second lattice matrix corresponding to a second surface structure of a second material different from the first material in the real plane, and a conversion table;
a processor; and
a user interface device,
wherein the processor is configured to:
load the first and second lattice matrices from the memory device;
obtain a first complex lattice matrix corresponding to the first surface structure in a complex plane using the first lattice matrix;
obtain a second complex lattice matrix corresponding to the second surface structure in the complex plane using the second lattice matrix;
obtain a lattice constant ratio of the first complex lattice matrix to the second complex lattice matrix;
obtain phase information and a conversion matrix for the given lattice constant ratio from the conversion table;
obtain common supercell information between the first and second materials from the lattice constant ratio, the phase information, and the conversion matrix; and
output the common supercell information through the user interface device.

11. The electronic device of claim 10, wherein the first surface structure of the first material and the second surface structure of the second material have a resemblance relationship.

12. The electronic device of claim 11, wherein a relationship between the first and second complex lattice matrices is defined by an eigenvalue, and the eigenvalue includes the lattice constant ratio and the phase information.

13. The electronic device of claim 10, wherein the first and the second materials have cubic structures, the lattice constant ratio is √(m²+n²), the phase information is arctan(n/m), and the conversion matrix includes a first conversion element value (e₁₁), a second conversion element value (e₁₂), a third conversion element value (e₂₁), and a fourth conversion element value (e₂₂), the first to fourth conversion element values (e₁₁, e₁₂, e₂₁, e₂₂) correspond to m, n, −n, and m, respectively, where n and m are arbitrary integers.

14. The electronic device of claim 10, wherein the first and the second materials have a hexagonal structure, the lattice constant ratio is √(m²—mn+n²), the phase information is arctan((√(3)*n)/(2m−n)), and the conversion matrix includes a first conversion element value (e₁₁), a second conversion element value (e₁₂), a third conversion element value (e₂₁), and a fourth conversion element value (e₂₂), the first to fourth conversion element values (e₁₁, e₁₂, e₂₁, e₂₂) correspond to m, n, −n, and m−n, respectively, where n and m are arbitrary integers.

15. The electronic device of claim 10, wherein the conversion table is implemented as a table with the form of a Python list.
